(19) United States
(12) Patent Application Publication
(10) Pub. No.: US 2023/0191284 A1
(43) Pub. Date: Jun. 22, 2023

(54) FULLY-AUTOMATIC PROTEIN PURIFICATION SYSTEM DEVICE AND USE THEREOF

(71) Applicant: THE INSTITUTE OF BIOPHYSICS OF THE CHINESE ACADEMY OF SCIENCES, Beijing (CN)

(72) Inventors: Xianjin OU, Beijing (CN); Xuemei LI, Beijing (CN)

(73) Assignee: THE INSTITUTE OF BIOPHYSICS OF THE CHINESE ACADEMY OF SCIENCES, Beijing (CN)

(21) Appl. No.: 17/926,952

(22) PCT Filed: May 18, 2021
(86) PCT No.: PCT/CN2021/094447

§ 371 Date: Nov. 21, 2022

(30) Foreign Application Priority Data
May 21, 2020 (CN) 202010434072.2

(51) Int. Cl. B01D 15/38; C07K 1/22; B01D 15/10

(52) U.S. Cl. CPC B01D 15/3804; C07K 1/22; B01D 15/10

(57) ABSTRACT

A fully-automatic protein purification system device includes a chromatography unit, a first drive unit, a connecting pipeline, a locating unit, a second drive unit, a first container, a second container, a first valve, a second valve, and a control unit. The fully-automatic protein purification system device can fully automate the protein chromatography purification with a simple device structure and a low cost, has low requirements for the quality of a sample solution, will not cause blockage of a pipeline, has a wide application range, can greatly improve the automation of protein chromatography purification in the biology field, and can reduce the manual investment.

### CROSS REFERENCE TO THE RELATED APPLICATIONS

This application is the national phase entry of International Application No. PCT/CN2021/094447, filed on May 18, 2021, which is based upon and claims priority to Chinese Patent Application No. 202010434072.2, filed on May 21, 2020, the entire contents of which are incorporated herein by reference.

### TECHNICAL FIELD

The present disclosure relates to the fields of chemical engineering and biology, particularly, to a fully-automatic protein purification system device and use thereof in the separation and purification such as affinity chromatography of a protein. cl BACKGROUND

Protein is an important research object and material in the field of scientific research and also acts as an important industrial product in the social life, such as insulin for treating diabetes. Protein separation and purification are essential operations in both scientific research and industrial production. Since genetic engineering technology is developed, a protein is usually prepared by constructing a recombinant cell by a recombinant genetic engineering means and then artificially controlling the enhanced expression of the protein. During the construction process of a recombinant expression cell, a polypeptide/protein sequence that can be specifically and reversibly adsorbed is added upstream or downstream of a target protein sequence, which can greatly facilitate the enrichment and purification of the target protein. The polypeptide sequence that can reversibly bind to a specific medium is usually called a tag. This protein purification method is called affinity chromatography and is one of the most common operations in the field of biology. A common purification tag for affinity chromatography is a sequence of 6 consecutive histidines, which is denoted as His*6. Specific affinity chromatography operations are as follows: First, a His*6 tag-containing target protein solution is driven by its gravity to flow through a chelated nickel ion medium, which is repeated 2 to 3 times to make the target protein fully bind to the medium. Second, after the target protein is specifically adsorbed, the other protein is removed with a buffer solution including low-concentration (usually 10 mM to 20 mM) imidazole. Finally, the chelated nickel ion medium is eluted with a buffer including high-concentration imidazole to obtain a high-purity target protein, whose purity can usually be 80% or higher.

At present, protein affinity chromatography operations are mainly conducted manually and have the following disadvantages: 1) The whole process lasts for several to dozens of hours, which is time-consuming and laborious. 2) The viscosity, concentration, and other properties of the protein sample solution vary each time, such that the flow rate at which the protein sample solution flows through the medium varies greatly, resulting in different affinity binding effects for a protein.

Chinese Patent Application CN201711448428.2 “Automatic Protein Purification Device for Gravity Column” discloses an automatic protein purification device, which relies on the gravity of a sample solution as a driving force. However, the disadvantage of the automatic protein purification device is that it is merely suitable for gravity chromatography columns. If a sample solution has too high of a concentration or a viscosity, the sample solution will flow too slowly or cause blockage, thereby affecting the operation effect. In addition, the device is a semi-automatic device and cannot achieve the full automation of resin binding of a protein, removal of other protein, and elution of a target protein.

The protein purification system AKTA™ of GE Healthcare can automatically achieve the adsorption, impurity removal, and elution of a protein. The system is usually powered by a high-end precision fluid pump, is provided with a sampling solenoid valve and a multi-position selective solenoid valve to achieve a liquid flow operation, and is also provided with a multi-wavelength ultraviolet (UV) detector to determine an operating status and an action switch. The system has the following two disadvantages: 1. The system is very expensive, where a model that can only achieve a non-automatic conventional protein purification operation usually costs between 400,000 yuan to 500,000 yuan, and a model that can achieve a fully-automatic protein purification operation is at a higher cost. 2. Key components of the system such as a fluid pump, a sampling valve, and a rotary valve are precision instruments and have high requirements for the quality of a sample solution. If a sample solution has a low quality, it easily causes blockage or leakage.

### SUMMARY

To solve the deficiencies of the prior art, an objective of the present disclosure is to provide a fully-automatic protein purification system device including a stepper motor, a two-way valve, a peristaltic pump, and a control system thereof, which can fully automate the protein chromatography purification with a simple device structure and a low cost, has low requirements for the quality of a sample solution, will not cause blockage of a pipeline, has a wide application range, can greatly improve the automation of protein chromatography purification in the biology field, and can reduce the manual investment.

To achieve the above objective, the present disclosure provides a fully-automatic protein purification system device, including a chromatography unit 0, a first drive unit, a connecting pipeline 2, a locating unit 3, a second drive unit, a first container 5, a second container 6, a first valve, a second valve, and a control unit. The connecting pipeline 2 has one end connected to the chromatography unit 0 and the other end connected to the locating unit 3. The second drive unit drives the locating unit 3. The first container 5 is connected to an upper part of the chromatography unit 0 with a pipeline through a first two-way valve, and the second container 6 is connected to the upper part of the chromatography unit 0 with a pipeline through a second two-way valve. The first drive unit drives a solution in the first container 5 to flow through the chromatography unit 0, and a liquid flowing out is collected in the second container 6 located below the connecting pipeline 2 through the connecting pipeline 2. The connecting pipeline 2 rotates with the locating unit 3, such that an outlet of the connecting pipeline 2 is located above the second container 6.

In an embodiment of the present disclosure, the first drive unit is a peristaltic pump 1.

In an embodiment of the present disclosure, the second drive unit is a motor 4.

In an embodiment of the present disclosure, the first valve is a first two-way valve 51.

In an embodiment of the present disclosure, the second valve is a second two-way valve 61.

In an embodiment of the present disclosure, the chromatography unit 0 includes a liquid level detector 11.

In an embodiment of the present disclosure, the chromatography unit 0 is a chromatography column.

In an embodiment of the present disclosure, the connecting pipeline 2 is a hose and preferably a silicone hose.

In an embodiment of the present disclosure, the locating unit 3 is a locating column; preferably, the locating unit is able to rotate 360°.

In an embodiment of the present disclosure, the motor 4 is a stepper motor.

In an embodiment of the present disclosure, the fully-automatic protein purification system device at least includes the first container 5 and the second container 6. Preferably, the fully-automatic protein purification system device further includes four other containers, that is, the fully-automatic protein purification system device includes 6 containers.

In an embodiment of the present disclosure, the fully-automatic protein purification system device further includes a third container 7, a fourth container 8, a fifth container 9, a sixth container 10, a third valve, a fourth valve, a fifth valve, and a sixth valve, where the third container 7, the fourth container 8, the fifth container 9, and the sixth container 10 are connected to the upper part of the chromatography unit 0 with pipelines through the third valve, the fourth valve, the fifth valve, and the sixth valve, respectively.

In an embodiment of the present disclosure, the third valve is a third two-way valve 71.

In an embodiment of the present disclosure, the fourth valve is a fourth two-way valve 81.

In an embodiment of the present disclosure, the fifth valve is a fifth two-way valve 91.

In an embodiment of the present disclosure, the sixth valve is a sixth two-way valve 101.

In an embodiment of the present disclosure, the two-way valve is a two-way solenoid valve.

In an embodiment of the present disclosure, all valves are normally-closed two-way valves, and are opened only after being powered up; preferably, at any time point, only one valve is opened and the remaining ones are closed.

In an embodiment of the present disclosure, when used for purifying a protein, the fully-automatic protein purification system device can fully automate the adsorption, washing, and elution of the protein.

In an embodiment of the present disclosure, a corresponding chromatography column can be added according to the specific needs of a user when the fully-automatic protein purification system device is in use.

The present disclosure has the following beneficial effects:

(1) The device of the present disclosure can fully automate the chromatography purification of a protein sample.

(2) The present disclosure adopts two or more containers to accommodate a sample solution and a cleaning solution, and the selection of different sample solutions can be simply achieved through the on/off of a fluid control valve (such as a solenoid valve).

(3) A selection of a flow direction of a liquid flowing out of the chromatography column can be simply determined through the rotation of only one drive unit (such as a stepper motor).

(4) The design of liquid level detection and delay time thereof can facilitate a switch action among different liquids to ensure that a chromatography medium will not be exhausted.

(5) A liquid is driven by a peristaltic pump, which is simple and reliable, has low requirements for the quality of a sample solution, and increases the application scope of the device.

### BRIEF DESCRIPTION OF THE DRAWINGS

FIG. 1 is a schematic structural diagram of the fully-automatic protein purification system device of the present disclosure.

FIG. 2 is a schematic diagram illustrating the working status of the fully-automatic protein purification system device of the present disclosure, where a silicone hose 2 is located directly above a second container 6.

### REFERENCE NUMERALS

0 represents a chromatography column; 1 represents a peristaltic pump; 2 represents a silicone hose; 3 represents a locating column; 4 represents a stepper motor; 5 represents a first container; 6 represents a second container; 7 represents a third container; 8 represents a fourth container; 9 represents a fifth container; 10 represents a sixth container; 51 represents a first two-way solenoid valve; 61 represents a second two-way solenoid valve; 71 represents a third two-way solenoid valve; 81 represents a fourth two-way solenoid valve; 91 represents a fifth two-way solenoid valve; 101 represents a sixth two-way solenoid valve; and 11 represents a liquid level detector.

### DETAILED DESCRIPTION OF THE EMBODIMENTS

The present disclosure is further described below in conjunction with specific examples, and these examples are implemented under the premise of the technical solutions of the present disclosure. It should be understood that these examples are provided merely to illustrate the present disclosure rather than to limit the scope of the present disclosure.

### EXAMPLE 1

### A Fully-Automatic Protein Purification System Device

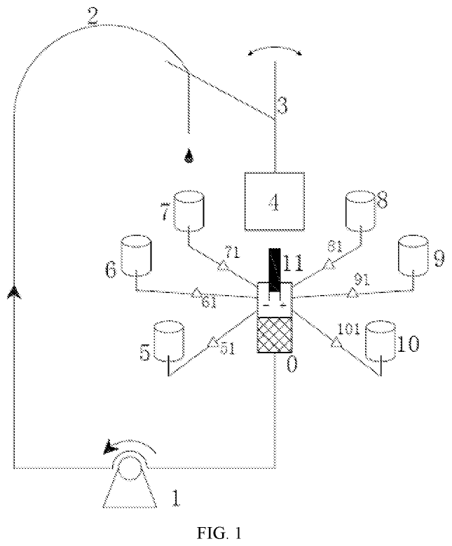

FIG. 1 shows the fully-automatic protein purification system device. The fully-automatic protein purification system device includes a chromatography column 0, a peristaltic pump 1, a silicone hose 2, a locating column 3, a stepper motor 4, a first container 5, a second container 6, a third container 7, a fourth container 8, a fifth container 9, a sixth container 10, a first two-way solenoid valve 51, a second two-way solenoid valve 61, a third two-way solenoid valve 71, a fourth two-way solenoid valve 81, a fifth two-way solenoid valve 91, a sixth two-way solenoid valve 101, a liquid level detector 11, and a circuit board control system thereof. The stepper motor 4 drives the locating column 3 connected thereto, such that the locating column can rotate 360° . The silicone hose 2 has one end connected to an outlet of the chromatography column 0, a middle part clamped on the peristaltic pump, and the other end fixed on a crossbar of the locating column. According to a set instruction, the silicone hose 2 rotates with the locating column 3, such that an outlet of the silicone hose 2 may be located above any one of the first container 5, the second container 6, the third container 7, the fourth container 8, the fifth container 9, and the sixth container 10 (the outlet is located above the third container 7 in FIG. 1).

The chromatography column 0 is divided into an upper part and a lower part, where the upper part of the chromatography column 0 communicates with a container through a pipeline, and the lower part of the chromatography column 0 (the grid part) is filled with an affinity medium. An outlet connecting pipeline is provided at the bottom of each of the first container 5, the second container 6, the third container 7, the fourth container 8, the fifth container 9, and the sixth container 10. The connecting pipeline is accordingly first connected to the first two-way solenoid valve 51, the second two-way solenoid valve 61, the third two-way solenoid valve 71, the fourth two-way solenoid valve 81, the fifth two-way solenoid valve 91, or the sixth two-way solenoid valve 101 and then connected to an upper interface of the chromatography column 0 through a pipeline. All solenoid valves are normally-closed solenoid valves and are opened only after being powered up. According to a preset instruction of an operation program, at any time point, only one solenoid valve is opened and the remaining ones are closed. A lower outlet of the chromatography column 0 is connected to the silicone hose 2, and the silicone hose 2 is controlled and powered by the peristaltic pump 1. According to a preset instruction, a solenoid valve in one of the first container 5, the second container 6, the third container 7, the fourth container 8, the fifth container 9, and the sixth container 10 is opened, the peristaltic pump drives a solution in a corresponding container to flow through the chromatography column 0, and a liquid flowing out is collected in a container located directly below the silicone hose 2 through the silicone hose 2.

A liquid level detector 11 is provided at an upper position with a specified height inside the chromatography column 0, which is configured to remind a liquid level through the conductivity of a solution. When a liquid flows normally, an AB metal probe above the liquid level detector (as shown in “+” and “−” below 11 in FIG. 1) is immersed in a sample solution, and due to the conductivity of the liquid, the detector will give a high electrical level signal, indicating that the solution is currently flowing normally. When a sample is about to be exhausted, a liquid level during chromatography slowly drops, and at a specified time point, the detector is exposed above the liquid level, and two electrodes on the probe are disconnected, which will give the system a low electrical level signal, indicating that the current liquid is about to be exhausted and the program is about to enter the next stage.

### EXAMPLE 2

### Control Parameter D esign

The following three operating parameters can be mainly set for the device of the present disclosure: binding times N, delay time T (s), and liquid flow rate. N represents the number of times a sample solution flows through a chromatography column, which is actually the number of times a medium adsorbs a sample. During manual protein chromatography purification, it is usually enough to conduct resin binding 2 times, and thus N is directly set to 2. The delay time T (s) means that, during an operation of the device, when the electrodes on the AB metal probe of the liquid level detector are just exposed above the liquid level and the system will receive a signal for switching from a high electrical level to a low electrical level, it actually still takes T (s) for the liquid level inside the chromatography column 0 in FIG. 1 to drop to an upper position of an adsorption medium (indicating that the sample is exhausted), and then the next operation is implemented, such as solenoid valve switching. The delay time T needs to be set according to specific parameters of the device, which is related to the liquid flow rate, the inner diameter of the chromatography column, and the height of the liquid level detector. The setting value of the delay time T is determined according to measured experimental results. The liquid flow rate is expressed as an amount (mL) of a liquid flowing through per minute and is related to the rotational speed of the peristaltic pump 1 and the size of the corresponding hose 2, which can be set according to the needs of a user and can also be determined according to the actual measurement results.

### EXAMPLE 3

### Protein affinity adsorption

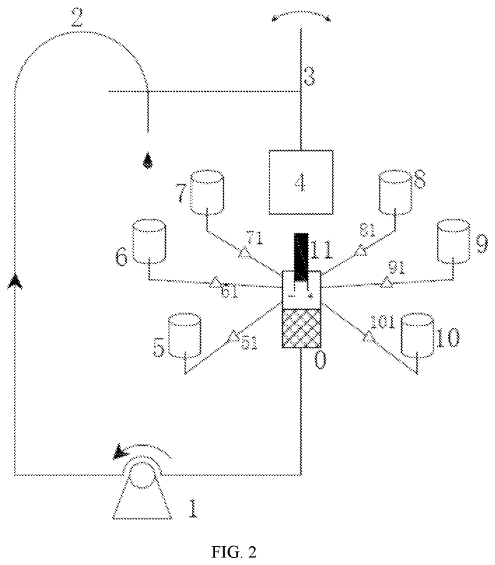

Escherichia coli (E. coli) cells carrying an affinity purification tag protein His*6 were subjected to ultrasonic disruption and then centrifuged at 16,000 g for 30 min to obtain 50 mL of a supernatant, and the supernatant was placed in the first container 5 in FIG. 2. The binding times N was set to 2, the flow rate was set to 1.5 mL/min, and the delay time T was equal to 30 s. A chromatography column filled with a nickel medium was well equilibrated with a 50 mM Tris protein buffer (pH=8) in advance. After the “Adsorption” mode was selected and the automatic program was started, the first two-way solenoid valve 51 connected to the first container 5 was powered up and was allowed to communicate with a pipeline, and the silicone hose 2 was located directly above the second container 6. The peristaltic pump was started to drive a fresh protein solution in the first container 5 to continuously flow through the solenoid valve 51 and then flow through the chromatography column 0 with an affinity medium, and a liquid flowing out after the adsorption was collected in the second container 6 through the silicone hose 2. The liquid level detector 11 was always in a high electrical level status throughout the adsorption process. At about 29 min, the sample solution was about to be exhausted and the liquid level detector was switched to a low electrical level; 30 s later, the system closed the first two-way solenoid valve 51 and opened the second two-way solenoid valve 61, and then the locating column was rotated such that the silicone hose 2 fixed on the locating column was located directly above the first container 5 to collect a sample solution obtained after the second resin binding in the next step. The second resin binding of the protein sample solution was then started, and within a few seconds after the second two-way solenoid valve 61 was opened, the liquid level detector was immersed in the sample solution once again, and the system was switched to a high electrical level. About 30 min later, the liquid level detector was exposed above the liquid level once again and the high electrical level was switched to a low electrical level, such that the second adsorption operation of the protein sample solution was completed, and then all solenoid valves and the peristaltic pump were closed. The entire process was fully automated without human intervention.

### EXAMPLE 4

### Protein Affinity Adsorption, Impurity Removal, and Elution

A resin binding operation of a protein sample was exactly the same as Example 2 (the binding times N was set to 2, the flow rate was set to 1.5 mL/min, and the delay time T was equal to 30 s), and the “purification” mode was selected. After the resin binding was conducted twice, the non-specifically-adsorbed other protein was first washed off with 100 mL of a washing buffer (50 mM Tris buffer, 20 mM imidazole, pH=8) placed in the third container 7, the target protein was eluted with 50 mL of an elution buffer (50 mM Tris buffer, 300 mM imidazole, pH=8) placed in the fourth container 8, and other settings were the same as Example 1.

When the second resin binding was about to be completed (as shown in Example 2), the liquid level detector was switched from a high electrical level to a low electrical level; 30 s later, the system closed the second two-way solenoid valve 61 and opened the third two-way solenoid valve 71, and the silicone hose 2 was driven by the stepper motor 4 such that the silicone hose 2 was located directly above the sixth container 10 to collect a waste liquid resulting from the washing of the chromatography column. The automation of washing-off of the other protein on the chromatography column was started, such that the washing buffer in the third container 7 was allowed to flow through the third two-way solenoid valve 71 and then flow through the chromatography column 0 with the affinity medium, and a liquid flowing out after the washing-off was collected in the sixth container 10 through the silicone hose 2. The liquid level detector was always immersed in a liquid and was at a high electrical level throughout the washing process. About 66 min later, the liquid level detector was switched to a low electrical level, and 30 s later, the system completed the other protein removal operation of the chromatography column.

The system closed the third two-way solenoid valve 71 and opened the fourth two-way solenoid valve 81, and the silicone hose 2 was driven by the stepper motor 4 such that the silicone hose 2 was located directly above the fifth container 9 to collect a target protein. The continuous elution of the target protein for about 30 min was started, such that the elution buffer in the fourth container 8 was allowed to flow through the fourth two-way solenoid valve 81 and then flow through the chromatography column 0 with the affinity medium, and a liquid flowing out after the elution was collected in the fifth container 9 through the silicone hose 2 to obtain an eluted protein sample solution. Throughout the elution process, 30 s after the liquid level detector was switched from a high electrical level to a low electrical level once again, the system closed the peristaltic pump and all solenoid valves.

Through the above steps, the system fully automated the adsorption, washing, and elution of the target protein.

What is claimed is:

1. A fully-automatic protein purification system device, comprising a chromatography unit, a first drive unit, a connecting pipeline, a locating unit, a second drive unit, a first container, a second container, a first valve, a second valve, and a control unit, wherein the connecting pipeline has a first end connected to the chromatography unit and a second end connected to the locating unit; the second drive unit drives the locating unit; the first container is connected to an upper part of the chromatography unit with a first pipeline through a first two-way valve, and the second container is connected to the upper part of the chromatography unit with a second pipeline through a second two-way valve; the first drive unit drives a solution in the first container to flow through the chromatography unit, and a liquid flowing out is collected in the second container located below the connecting pipeline through the connecting pipeline; the connecting pipeline rotates with the locating unit, such that an outlet of the connecting pipeline is located above the second container.

2. The fully-automatic protein purification system device according to claim 1, wherein the chromatography unit comprises a liquid level detector.

3. The fully-automatic protein purification system device according to claim 1, wherein the chromatography unit is a chromatography column.

4. The fully-automatic protein purification system device according to claim 1, wherein the connecting pipeline is a hose.

5. The fully-automatic protein purification system device according to claim 1, wherein the locating unit is a locating column; and the locating unit is configured to rotate 360°.

6. The fully-automatic protein purification system device according to claim 1, wherein the second drive unit is a stepper motor.

7. The fully-automatic protein purification system device according to claim 1, further comprising: a third container, a fourth container, a fifth container, a sixth container.

8. The fully-automatic protein purification system device according to claim 1, further comprising: a third container, a fourth container, a fifth container, a sixth container, a third valve, a fourth valve, a fifth valve, and a sixth valve, wherein the third container, the fourth container, the fifth container, and the sixth container are connected to the upper part of the chromatography unit with third pipelines through the third valve, the fourth valve, the fifth valve, and the sixth valve, respectively;
the third valve is a third two-way valve;
the fourth valve is a fourth two-way valve;
the fifth valve is a fifth two-way valve;
the sixth valve is a sixth two-way valve; and
each of the first two-way valve, the second two-way valve, the third two-way valve, the fourth two-way valve, the fifth two-way valve, and the sixth two-way valve is a two-way solenoid valve.

9. The fully-automatic protein purification system device according to claim 1, wherein the first valve and the second valve are normally-closed valves and are opened only after being powered up; and at any time point, only one valve of the first valve and the second valve is opened and the remaining one of the first valve and the second valve is closed.

10. A method of use of the fully-automatic protein purification system device according to claim 1 in a purification of a protein.

11. The fully-automatic protein purification system device according to claim 1, wherein the first drive unit is a peristaltic pump.

12. The fully-automatic protein purification system device according to claim 1, wherein the the second drive unit is a motor.

13. The fully-automatic protein purification system device according to claim 1, wherein the the first valve is the first two-way valve.

14. The fully-automatic protein purification system device according to claim 1, wherein the the second valve is the second two-way valve.

15. The fully-automatic protein purification system device according to claim 2, wherein the the chromatography unit is a chromatography column.

16. The fully-automatic protein purification system device according to claim 2, wherein the connecting pipeline is a hose.

17. The fully-automatic protein purification system device according to claim 3, wherein the connecting pipeline is a hose.

18. The fully-automatic protein purification system device according to claim 4, wherein the horse is a silicone hose.

19. The fully-automatic protein purification system device according to claim 3, wherein the locating unit is a locating column; and the locating unit is configured to rotate 360°.

20. The fully-automatic protein purification system device according to claim 4, wherein the locating unit is a locating column; and the locating unit is configured to rotate 360°.
